(19) United States
(12) Patent Application Publication
(10) Pub. No.: US 2022/0386544 A1
(43) Pub. Date: Dec. 8, 2022

(54) HYDRATION AND SANITIZATION SYSTEM FOR AN INDOOR GARDENING APPLIANCE

(71) Applicant: Haier US Appliance Solutions, Inc., Wilmington, DE (US)

(72) Inventors: Hyesoo An, Seoul (KR); Je Kwon Yoon, Seongnam (KR)

(21) Appl. No.: 17/339,259

(22) Filed: Jun. 4, 2021

(51) Int. Cl. A01G 31/02; A61L 2/00; C25B 1/22

(52) U.S. Cl. CPC A01G 31/02; A61L 2/0088; C25B 1/22; A61L 2202/15; A61L 2101/20

(57) ABSTRACT

An indoor gardening appliance includes a grow module positioned within a grow chamber for receiving one or more plant pods. The indoor gardening system includes a hydration and sanitization system that includes a water supply for providing a flow of water into a mixing tank that is periodically discharged through a discharge nozzle to hydrate and provide nutrients to plants. A sanitization assembly includes an electrolytic hypochlorous acid generator that is fluidly coupled to the mixing tank for selectively generating hypochlorous acid that helps sanitize plants within the grow chamber.

### FIELD OF THE INVENTION

The present subject matter relates generally to systems for gardening plants indoors, and more particularly, to systems and methods for hydrating and sanitizing plants within an indoor gardening appliance.

### BACKGROUND OF THE INVENTION

Conventional indoor garden centers include a cabinet defining a grow chamber having a number of trays or racks positioned therein to support seedlings or plant material, e.g., for growing herbs, vegetables, or other plants in an indoor environment. In addition, such indoor garden centers may include an environmental control system that maintains the growing chamber at a desired temperature or humidity. Certain indoor garden centers may also include hydration systems for watering the plants and/or artificial lighting systems that provide the light necessary for such plants to grow.

Notably, plants positioned within conventional indoor gardens centers may frequently be contaminated by an external source, resulting in the buildup of bacteria, germs, fungus, etc. For example, a user may frequently interact with and contact growing plants with their hands that are contaminated with various germs and bacteria. Certain conventional indoor gardening appliances may include internal cleaning procedures or systems, but these systems commonly rely on harsh chemicals, ozone generation systems, or other sanitization sources that may be hazardous to humans, whether through direct exposure or secondary exposure by consuming plants sanitized using such sanitization sources.

Accordingly, an improved indoor garden center would be useful. More particularly, an indoor garden center with a hydration and sanitization system that facilitates improved plant growth in a sanitized environment would be particularly beneficial.

### BRIEF DESCRIPTION OF THE INVENTION

Aspects and advantages of the invention will be set forth in part in the following description, or may be apparent from the description, or may be learned through practice of the invention.

In one exemplary embodiment, an indoor gardening appliance is provided including a liner positioned within a cabinet and defining a grow chamber, a grow module mounted within the liner and defining a plurality of apertures for receiving one or more plant pods, and a sanitization assembly. The sanitization assembly includes a water supply for providing a flow of water, an electrolytic hypochlorous acid generator for receiving the flow of water and generating a hypochlorous acid solution, and a discharge nozzle for selectively discharging the hypochlorous acid solution into the grow chamber.

In another exemplary embodiment, a hydration system for a gardening appliance is provided. The gardening appliance includes a liner positioned within a cabinet and defining a grow chamber and a grow module mounted within the liner and defining a plurality of apertures for receiving one or more plant pods. The hydration system includes a water supply for providing a flow of water, a mixing tank fluidly coupled to the water supply through a water supply conduit, and a sanitization assembly. The sanitization assembly includes an electrolytic hypochlorous acid generator fluidly coupled to the mixing tank for receiving the flow of water and generating a hypochlorous acid solution and a discharge nozzle for selectively discharging the hypochlorous acid solution into the grow chamber.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures.

FIG. 1 provides a perspective view of a gardening appliance according to an exemplary embodiment of the present subject matter.

FIG. 2 depicts a front view of the exemplary gardening appliance of FIG. 1 with the doors open according to an exemplary embodiment of the present subject matter.

FIG. 3 is a cross sectional view of the exemplary gardening appliance of FIG. 1, taken along Line 3-3 from FIG. 2 with an internal divider removed for clarity.

FIG. 4 is a top perspective view of the exemplary gardening appliance of FIG. 1, with the top panel of the cabinet removed to reveal a rotatable grow module according to an exemplary embodiment of the present subject matter.

FIG. 5 provides a perspective cross sectional view of the exemplary gardening appliance of FIG. 1 according to another exemplary embodiment of the present subject matter.

FIG. 6 provides a perspective view of the grow module of the exemplary gardening appliance of FIG. 1 according to another exemplary embodiment of the present subject matter.

FIG. 7 provides a perspective cross sectional view of the exemplary grow module of FIG. 6 according to another exemplary embodiment of the present subject matter.

FIG. 8 provides a top cross-sectional view of the exemplary grow module of FIG. 6 according to another exemplary embodiment of the present subject matter.

FIG. 9 provides a schematic view of a hydration and sanitization system of the exemplary gardening appliance of FIG. 1 according to an exemplary embodiment of the present subject matter.

FIG. 10 provides a schematic view of an electrolytic hypochlorous acid generator according to one or more embodiments of the present disclosure.

FIG. 11 provides a schematic view of an electrolytic hypochlorous acid generator according to one or more additional embodiments of the present disclosure.

Repeat use of reference characters in the present specification and drawings is intended to represent the same or analogous features or elements of the present invention.

### DETAILED DESCRIPTION OF THE INVENTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

As used herein, terms of approximation, such as “approximately,” “substantially,” or “about,” refer to being within a ten percent (10%) margin of error of the stated value. Moreover, as used herein, the terms “first,” “second,” and “third” may be used interchangeably to distinguish one component from another and are not intended to signify location or importance of the individual components. The terms “upstream” and “downstream” refer to the relative direction with respect to fluid flow in a fluid pathway. For example, “upstream” refers to the direction from which the fluid flows, and “downstream” refers to the direction to which the fluid flows.

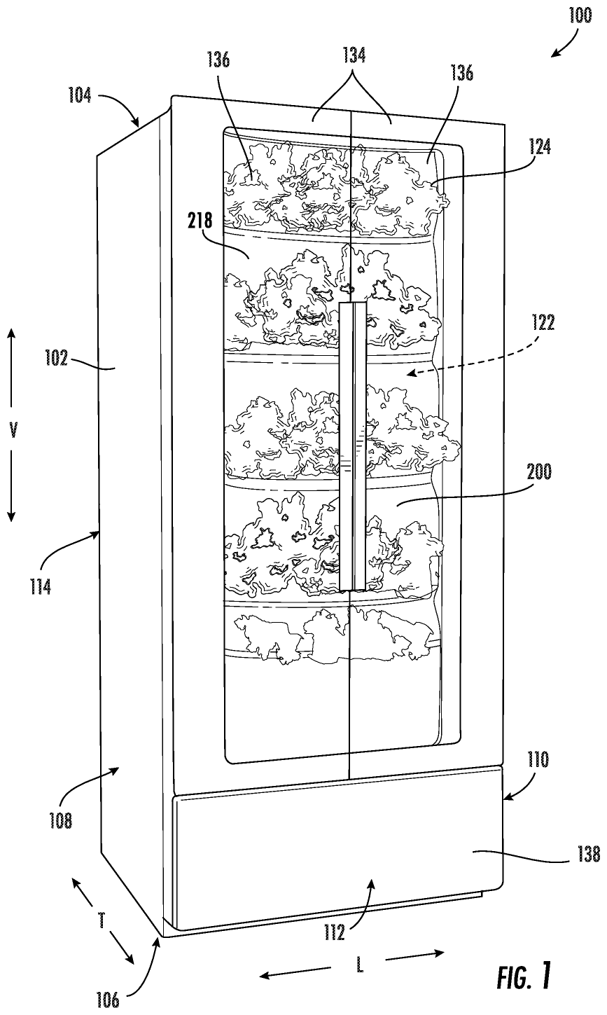

FIG. 1 provides a front view of a gardening appliance 100 according to an exemplary embodiment of the present subject matter. According to exemplary embodiments, gardening appliance 100 may be used as an indoor garden center for growing plants. It should be appreciated that the embodiments described herein are intended only for explaining aspects of the present subject matter. Variations and modifications may be made to gardening appliance 100 while remaining within the scope of the present subject matter.

Gardening appliance 100 includes a housing or cabinet 102 that extends between a top 104 and a bottom 106 along a vertical direction V, between a first side 108 and a second side 110 along a lateral direction L, and between a front side 112 and a rear side 114 along a transverse direction T. Each of the vertical direction V, lateral direction L, and transverse direction T are mutually perpendicular to one another and form an orthogonal direction system.

Gardening appliance 100 may include an insulated liner 120 positioned within cabinet 102. Liner 120 may at least partially define a temperature controlled chamber, referred to herein generally as a grow chamber 122, within which plants 124 may be grown. Although gardening appliance 100 is referred to herein as growing plants 124, it should be appreciated that other organisms or living things may be grown or stored in gardening appliance 100. For example, algae, fungi (e.g., including mushrooms), or other living organisms may be grown or stored in gardening appliance 100. The specific application described herein is not intended to limit the scope of the present subject matter.

Cabinet 102, or more specifically, liner 120 may define a substantially enclosed back region or portion 130. In addition, cabinet 102 and liner 120 may define a front opening, referred to herein as front display opening 132, through which a user of gardening appliance 100 may access grow chamber 122, e.g., for harvesting, planting, pruning, or otherwise interacting with plants 124. According to an exemplary embodiment, enclosed back portion 130 may be defined as a portion of liner 120 that defines grow chamber 122 proximate rear side 114 of cabinet 102. In addition, front display opening 132 may generally be positioned proximate or coincide with front side 112 of cabinet 102.

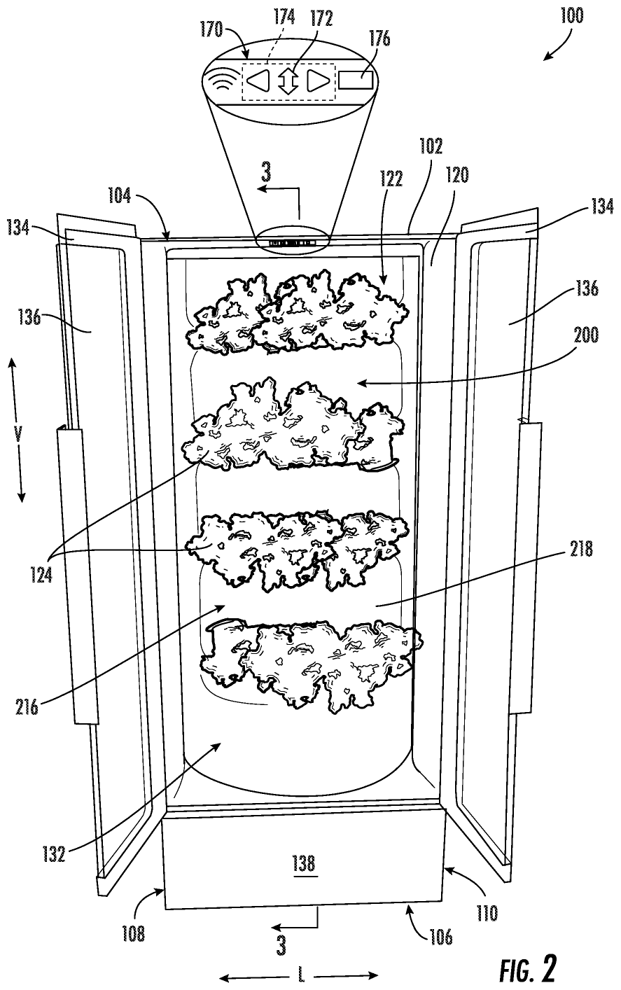

Gardening appliance 100 may further include one or more doors 134 that are rotatably mounted to cabinet 102 for providing selective access to grow chamber 122. For example, FIG. 1 illustrates doors 134 in the closed position such that they may help insulate grow chamber 122. By contrast, FIG. 2 illustrates doors 134 in the open positioned for accessing grow chamber 122 and plants 124 stored therein. Doors 134 may further include a transparent window 136 through which a user may observe plants 124 without opening doors 134.

Although doors 134 are illustrated as being rectangular and being mounted on front side 112 of cabinet 102 in FIGS. 1 and 2, it should be appreciated that according to alternative embodiments, doors 134 may have different shapes, mounting locations, etc. For example, doors 134 may be curved, may be formed entirely from glass, etc. In addition, doors 134 may have integral features for controlling light passing into and/or out of grow chamber 122, such as internal louvers, tinting, UV treatments, polarization, etc. One skilled in the art will appreciate that other chamber and door configurations are possible and within the scope of the present invention.

According to the illustrated embodiment, cabinet 102 further defines a drawer 138 positioned proximate bottom 106 of cabinet 102 and being slidably mounted to cabinet 102 for providing convenient storage for plant nutrients, system accessories, water filters, etc. In addition, behind drawer 138 is a mechanical compartment 140 for receipt of an environmental control system including a sealed system for regulating the temperature within grow chamber 122, as described in more detail below.

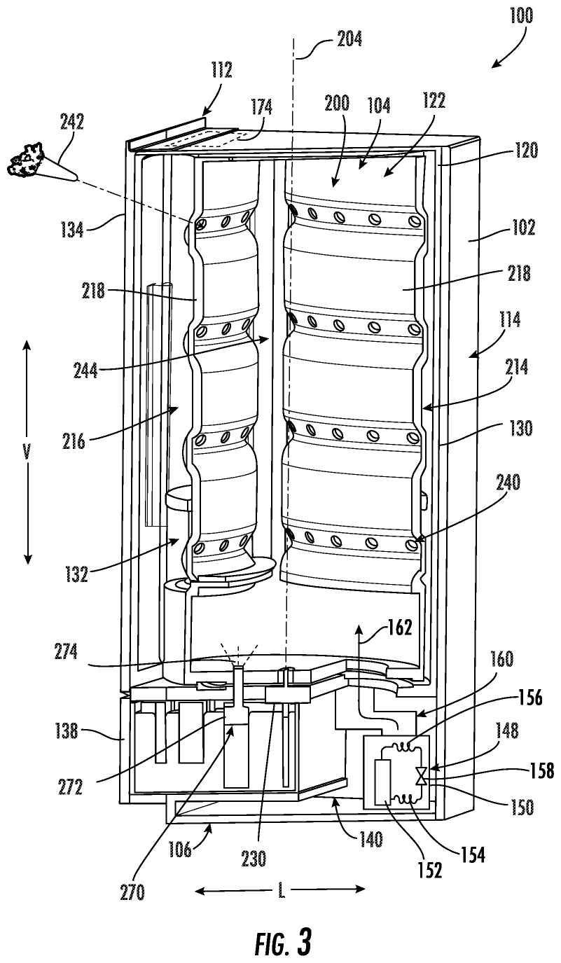

FIG. 3 provides a schematic view of certain components of an environmental control system 148 that may be used to regulate a temperature within grow chamber 122. Specifically, environmental control system 148 may include a sealed system 150, a duct system 160, and a hydration system 270, or any other suitable components or subsystems for regulating an environment within grow chamber 122, e.g., for facilitating improved or regulated growth of plants 124 positioned therein. Specifically, FIG. 3 illustrates sealed system 150 within mechanical compartment 140. Although an exemplary sealed system is illustrated and described herein, it should be appreciated that variations and modifications may be made to sealed system 150 while remaining within the scope of the present subject matter. For example, sealed system 150 may include additional or alternative components, different ducting configurations, etc.

As shown, sealed system 150 includes a compressor 152, a first heat exchanger or evaporator 154 and a second heat exchanger or condenser 156. As is generally understood, compressor 152 is generally operable to circulate or urge a flow of refrigerant through sealed system 150, which may include various conduits which may be utilized to flow refrigerant between the various components of sealed system 150. Thus, evaporator 154 and condenser 156 may be between and in fluid communication with each other and compressor 152.

During operation of sealed system 150, refrigerant flows from evaporator 154 and to compressor 152, and compressor 152 is generally configured to direct compressed refrigerant from compressor 152 to condenser 156. For example, refrigerant may exit evaporator 154 as a fluid in the form of a superheated vapor. Upon exiting evaporator 154, the refrigerant may enter compressor 152, which is operable to compress the refrigerant. Accordingly, the pressure and temperature of the refrigerant may be increased in compressor 152 such that the refrigerant becomes a more superheated vapor.

Condenser 156 is disposed downstream of compressor 152 and is operable to reject heat from the refrigerant. For example, the superheated vapor from compressor 152 may enter condenser 156 and transfer energy to air surrounding condenser 156 (e.g., to create a flow of heated air). In this manner, the refrigerant condenses into a saturated liquid and/or liquid vapor mixture. A condenser fan (not shown) may be positioned adjacent condenser 156 and may facilitate or urge the flow of heated air across the coils of condenser 156 (e.g., from ambient atmosphere) in order to facilitate heat transfer.

According to the illustrated embodiment, an expansion device or a variable electronic expansion valve 158 may be further provided to regulate refrigerant expansion. During use, variable electronic expansion valve 158 may generally expand the refrigerant, lowering the pressure and temperature thereof. In this regard, refrigerant may exit condenser 156 in the form of high liquid quality/saturated liquid vapor mixture and travel through variable electronic expansion valve 158 before flowing through evaporator 154. Variable electronic expansion valve 158 is generally configured to be adjustable, e.g., such that the flow of refrigerant (e.g., volumetric flow rate in milliliters per second) through variable electronic expansion valve 158 may be selectively varied or adjusted.

Evaporator 154 is disposed downstream of variable electronic expansion valve 158 and is operable to heat refrigerant within evaporator 154, e.g., by absorbing thermal energy from air surrounding the evaporator (e.g., to create a flow of cooled air). For example, the liquid or liquid vapor mixture refrigerant from variable electronic expansion valve 158 may enter evaporator 154. Within evaporator 154, the refrigerant from variable electronic expansion valve 158 receives energy from the flow of cooled air and vaporizes into superheated vapor and/or high quality vapor mixture. An air handler or evaporator fan (not shown) is positioned adjacent evaporator 154 and may facilitate or urge the flow of cooled air across evaporator 154 in order to facilitate heat transfer. From evaporator 154, refrigerant may return to compressor 152 and the vapor-compression cycle may continue.

As explained above, environmental control system 148 includes a sealed system 150 for providing a flow of heated air or a flow cooled air throughout grow chamber 122 as needed. To direct this air, environmental control system 148 includes a duct system 160 for directing the flow of temperature regulated air, identified herein simply as flow of air 162 (see, e.g., FIG. 3). In this regard, for example, an evaporator fan can generate a flow of cooled air as the air passes over evaporator 154 and a condenser fan can generate a flow of heated air as the air passes over condenser 156.

These flows of air 162 are routed through a cooled air supply duct and/or a heated air supply duct (not shown), respectively. In this regard, it should be appreciated that environmental control system 148 may generally include a plurality of ducts, dampers, diverter assemblies, and/or air handlers to facilitate operation in a cooling mode, in a heating mode, in both a heating and cooling mode, or any other mode suitable for regulating the environment within grow chamber 122. It should be appreciated that duct system 160 may vary in complexity and may regulate the flows of air from sealed system 150 in any suitable arrangement through any suitable portion of grow chamber 122.

Gardening appliance 100 may include a control panel 170. Control panel 170 includes one or more input selectors 172, such as e.g., knobs, buttons, push buttons, touchscreen interfaces, etc. In addition, input selectors 172 may be used to specify or set various settings of gardening appliance 100, such as e.g., settings associated with operation of sealed system 150. Input selectors 172 may be in communication with a processing device or controller 174. Control signals generated in or by controller 174 operate gardening appliance 100 in response to input selectors 172. Additionally, control panel 170 may include a display 176, such as an indicator light or a screen. Display 176 is communicatively coupled with controller 174 and may display information in response to signals from controller 174. Further, as will be described herein, controller 174 may be communicatively coupled with other components of gardening appliance 100, such as e.g., one or more sensors, motors, or other components.

As used herein, “processing device” or “controller” may refer to one or more microprocessors or semiconductor devices and is not restricted necessarily to a single element. The processing device can be programmed to operate gardening appliance 100. The processing device may include, or be associated with, one or more memory elements (e.g., non-transitory storage media). In some such embodiments, the memory elements include electrically erasable, programmable read only memory (EEPROM). Generally, the memory elements can store information accessible processing device, including instructions that can be executed by processing device. Optionally, the instructions can be software or any set of instructions and/or data that when executed by the processing device, cause the processing device to perform operations.

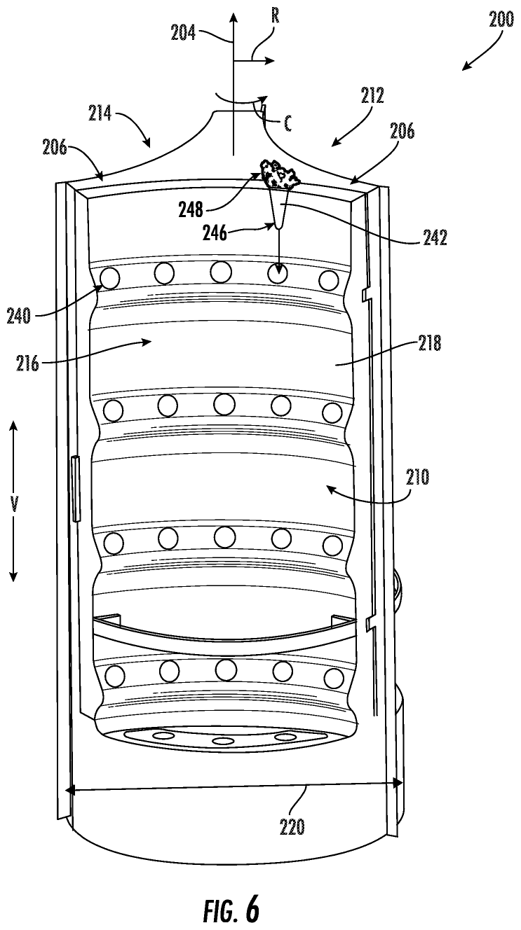

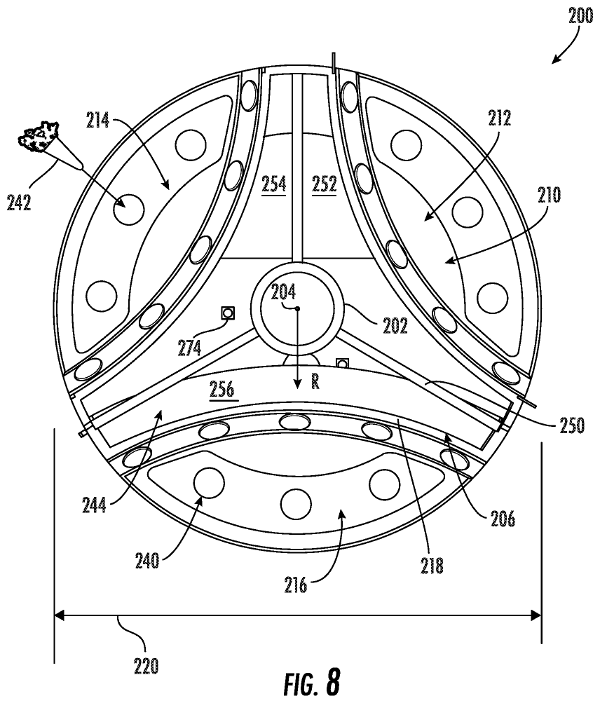

Referring now generally to FIGS. 1 through 8, gardening appliance 100 generally includes a rotatable carousel, referred to herein as a grow module 200 that is mounted within liner 120, e.g., such that it is within grow chamber 122. As illustrated, grow module 200 includes a central hub 202 that extends along and is rotatable about a central axis 204. Specifically, according to the illustrated embodiment, central axis 204 is parallel to the vertical direction V. However, it should be appreciated that central axis 204 could alternatively extend in any suitable direction, e.g., such as the horizontal direction. In this regard, grow module 200 generally defines an axial direction, i.e., parallel to central axis 204, a radial direction R that extends perpendicular to central axis 204, and a circumferential direction C that extends around central axis 204 (e.g. in a plane perpendicular to central axis 204).

Grow module 200 may further include a plurality of partitions 206 that extend from central hub 202 substantially along the radial direction R. In this manner, grow module 200 defines a plurality of chambers, referred to herein generally by reference numeral 210, by dividing or partitioning grow chamber 122. Referring specifically to a first embodiment of grow module 200 illustrated in FIGS. 1 through 8, grow module 200 includes three partitions 206 to define a first chamber 212, a second chamber 214, and a third chamber 216, which are circumferentially spaced relative to each other. In general, as grow module 200 is rotated within grow chamber 122, the plurality of chambers 210 define substantially separate and distinct growing environments, e.g., for growing plants 124 having different growth needs.

More specifically, partitions 206 may extend from central hub 202 to a location immediately adjacent liner 120. Although partitions 206 are described as extending along the radial direction, it should be appreciated that they need not be entirely radially extending. For example, according to the illustrated embodiment, the distal ends of each partition is joined with an adjacent partition using an arcuate wall 218, which is generally used to support plants 124.

Notably, it is desirable according to exemplary embodiments to form a substantial seal between partitions 206 and liner 120. Therefore, according to an exemplary embodiment, grow module 200 may define a grow module diameter 220 (e.g., defined by its substantially circular footprint formed in a horizontal plane). Similarly, enclosed back portion 130 of liner 120 may be substantially cylindrical and may define a liner diameter 222. In order to prevent a significant amount of air from escaping between partitions 206 and liner 120, liner diameter 222 may be substantially equal to or slightly larger than grow module diameter 220.

According to still other embodiments, grow module 200 may include one or more sealing elements 224 positioned on a radially distal end of each of partitions 206. In this regard, sealing elements 224 may extend from partitions 206 toward liner 120 to contact and seal against liner 120. For example, according to the illustrated embodiment, sealing elements 224 are wiper blades formed from silicone or another suitably resilient material. Thus, as grow module 200 rotates, sealing elements 224 slide against liner 120 to substantially seal each of the plurality of chambers 210. It should be appreciated that as used herein, the term “substantial seal” and the like is not intended to refer to a perfectly airtight junction. Instead, this term is generally used to refer to an environment which may be regulated independently of adjacent environments to a reasonable degree. For example, if plants 124 and the first chamber 212 prefer a 10° F. increase in temperature relative to plants 124 and second chamber 214, the substantial seal between these two chambers may facilitate such temperature difference.

Referring now specifically to FIG. 3, gardening appliance 100 may further include a motor 230 or another suitable driving element or device for selectively rotating grow module 200 during operation of gardening appliance 100. In this regard, according to the illustrated embodiment, motor 230 is positioned below grow module 200, e.g., within mechanical compartment 140, and is operably coupled to grow module 200 along central axis 204 for rotating grow module 200.

As used herein, “motor” may refer to any suitable drive motor and/or transmission assembly for rotating grow module 200. For example, motor 230 may be a brushless DC electric motor, a stepper motor, or any other suitable type or configuration of motor. For example, motor 230 may be an AC motor, an induction motor, a permanent magnet synchronous motor, or any other suitable type of AC motor. In addition, motor 230 may include any suitable transmission assemblies, clutch mechanisms, or other components.

According to an exemplary embodiment, motor 230 may be operably coupled to controller 174, which is programmed to rotate grow module 200 according to predetermined operating cycles, based on user inputs (e.g. via touch buttons 172), etc. In addition, controller 174 may be communicatively coupled to one or more sensors, such as temperature or humidity sensors, positioned within the various chambers 210 for measuring temperatures and/or humidity, respectively. Controller 174 may then operate motor 230 in order to maintain desired environmental conditions for each of the respective chambers 210. For example, as will be described in more detail below, gardening appliance 100 includes features for providing certain locations of gardening appliance 100 with light, temperature control, proper moisture, nutrients, and other requirements for suitable plant growth. Motor 230 may be used to position specific chambers 210 where needed to receive such growth requirements.

According to an exemplary embodiment, such as where three partitions 206 form three chambers 212-216, controller 174 may operate motor 230 to index grow module 200 sequentially through a number of preselected positions. More specifically, motor 230 may rotate grow module 200 in a counterclockwise direction (e.g. when viewed from a top of grow module 200) in 120° increments to move chambers 210 between sealed positions and display positions. As used herein, a chamber 210 is considered to be in a “sealed position” when that chamber 210 is substantially sealed between grow module 200 (i.e., central hub 202 and adjacent partitions 206) and liner 120. By contrast, a chamber 210 is considered to be in a “display position” when that chamber 210 is at least partially exposed to front display opening 132, such that a user may access plants 124 positioned within that chamber 210.

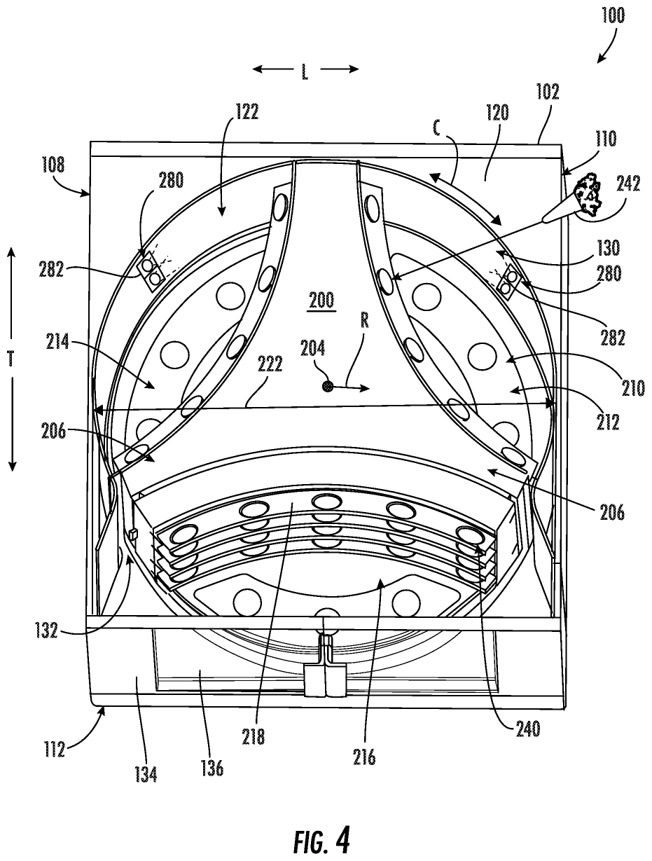

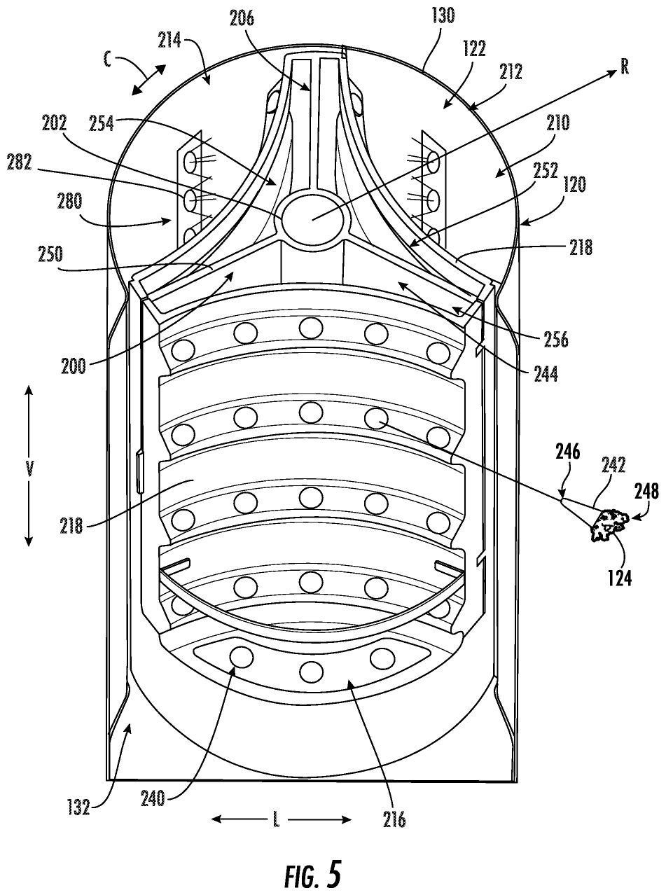

For example, as illustrated in FIGS. 4 and 5, first chamber 212 and second chamber 214 are both in a sealed position, whereas third chamber 216 is in a display position. As motor 230 rotates grow module 200 by 120 degrees in the counterclockwise direction, second chamber 214 will enter the display position, while first chamber 212 and third chamber 216 will be in the sealed positions. Motor 230 may continue to rotate grow module 200 in such increments to cycle grow chambers 210 between these sealed and display positions.

Referring now generally to FIGS. 4 through 8, grow module 200 will be described in more detail according to an exemplary embodiment of the present subject matter. As shown, grow module 200 defines a plurality of apertures 240 which are generally configured for receiving plant pods 242 into an internal root chamber 244. Plant pods 242 generally contain seedlings or other material for growing plants positioned within a mesh or other support structure through which roots of plants 124 may grow within grow module 200. A user may insert a portion of plant pod 242 (e.g., a seed end or root end 246) having the desired seeds through one of the plurality of apertures 240 into root chamber 244. A plant end 248 of the plant pod 242 may remain within grow chamber 210 such that plants 124 may grow from grow module 200 such that they are accessible by a user. In this regard, grow module 200 defines root chamber 244, e.g., within at least one of central hub 202 and the plurality of partitions 206. As will be explained below, water and other nutrients may be supplied to the root end 246 of plant pods 242 within root chamber 244. Notably, apertures 240 may be covered by a flat flapper seal (not shown) to prevent water from escaping root chamber 244 when no plant pod 242 is installed.

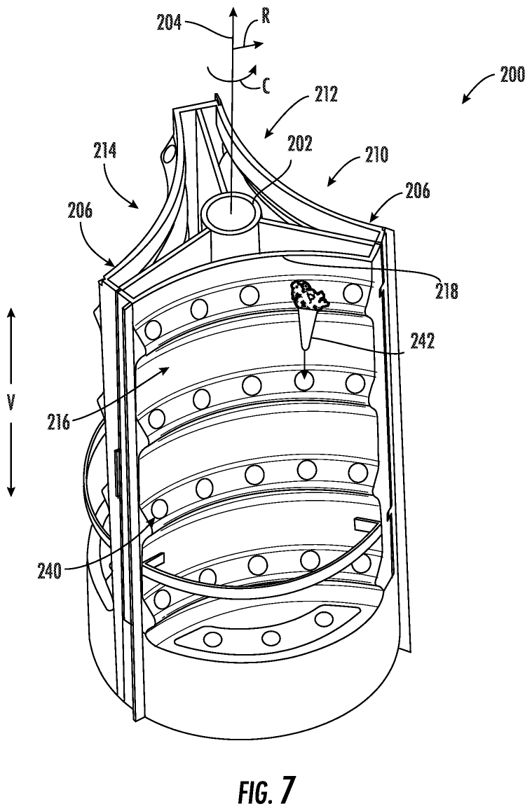

As best shown in FIGS. 5 and 7, grow module 200 may further include an internal divider 250 that is positioned within root chamber 244 to divide root chamber 244 into a plurality of root chambers, each of the plurality of root chambers being in fluid communication with one of the plurality of grow chambers 210 through the plurality of apertures 240. More specifically, according to the illustrated embodiment, internal divider 250 may divide root chamber 244 into a first root chamber 252, a second root chamber 254, and a third root chamber 256. According to an exemplary embodiment, first root chamber 252 may provide water and nutrients to plants 124 positioned in the first grow chamber 212, second root chamber 254 may provide water and nutrients to plants 124 positioned in the second grow chamber 214, and third root chamber 256 may provide water and nutrients to plants 124 positioned in the third grow chamber 216. In this manner, environmental control system 148 may control the temperature and/or humidity of each of the plurality of chambers 212-216 and the plurality of root chambers 252-256 independently of each other.

Environmental control system 148 may further include a hydration system 270 which is generally configured for providing water to plants 124 to support their growth. Specifically, according to the illustrated embodiment, hydration system 270 generally includes a water supply 272 and misting device 274 (e.g., such as a fine mist spray nozzle or nozzles). For example, water supply 272 may be a reservoir containing water (e.g., distilled water) or may be a direct connection municipal water supply. Misting device 274 may be positioned at a bottom of root chamber 244 and may be configured for charging root chamber 244 with mist for hydrating the roots of plants 124. Alternatively, misting devices 274 may pass through central hub 204 along the vertical direction V and periodically include a nozzle for spraying a mist or water into root chamber 244 or grow chamber 122. Because various plants 124 may require different amounts of water for desired growth, hydration system 270 may alternatively include a plurality of misting devices 274, e.g., all coupled to water supply 272, but being selectively operated to charge each of first root chamber 252, second root chamber 254, and third root chamber 256 independently of each other.

Notably, environmental control system 148 described above is generally configured for regulating the temperature and humidity (e.g., or some other suitable water level quantity or measurement) within one or all of the plurality of chambers 210 and/or root chambers 252-256 independently of each other. In this manner, a versatile and desirable growing environment may be obtained for each and every chamber 210.

Referring now for example to FIGS. 4 and 5, gardening appliance 100 may further include a light assembly 280 which is generally configured for providing light into selected grow chambers 210 to facilitate photosynthesis and growth of plants 124. As shown, light assembly 280 may include a plurality of light sources 282 stacked in an array, e.g., extending along the vertical direction V. For example, light sources 282 may be mounted directly to liner 120 within grow chamber 122, or may alternatively be positioned behind liner 120 such that light is projected through a transparent window or light pipe into grow chamber 122. The position, configuration, and type of light sources 282 described herein are not intended to limit the scope of the present subject matter in any manner.

Light sources 282 may be provided as any suitable number, type, position, and configuration of electrical light source(s), using any suitable light technology and illuminating in any suitable color. For example, according to the illustrated embodiment, light source 282 includes one or more light emitting diodes (LEDs), which may each illuminate in a single color (e.g., white LEDs), or which may each illuminate in multiple colors (e.g., multi-color or RGB LEDs) depending on the control signal from controller 174. However, it should be appreciated that according to alternative embodiments, light sources 282 may include any other suitable traditional light bulbs or sources, such as halogen bulbs, fluorescent bulbs, incandescent bulbs, glow bars, a fiber light source, etc.

According to an exemplary embodiment, light assembly 280 is positioned only within the enclosed back portion 130 of liner 120 such that only grow chambers 210 which are in a sealed position are exposed to light from light sources 282. Specifically, grow module 200 acts as a physical partition between light assemblies 280 and front display opening 132. In this manner, as illustrated in FIG. 5, no light may pass from first chamber 212 or second chamber 214 through grow module 200 and out front display opening 132. As grow module 200 rotates, two of the three grow chambers 210 will receive light from light assembly 280 at a time. According still other embodiments, a single light assembly may be used to reduce costs, whereby only a single grow chamber 210 will be lit at a single time.

Gardening appliance 100 and grow module 200 have been described above to explain an exemplary embodiment of the present subject matter. However, it should be appreciated that variations and modifications may be made while remaining within the scope of the present subject matter. For example, according to alternative embodiments, gardening appliance 100 may be a simplified to a two-chamber embodiment with a square liner 120 and a grow module 200 having two partitions 206 extending from opposite sides of central hub 202 to define a first grow chamber and a second grow chamber. According to such an embodiment, by rotating grow module 200 by 180 degrees about central axis 206, the first chamber may alternate between the sealed position (e.g., facing rear side 114 of cabinet 102) and the display position (e.g., facing front side 112 of cabinet 102). By contrast, the same rotation will move the second chamber from the display position to the sealed position.

According to still other embodiments, gardening appliance 100 may include a three chamber grow module 200 but may have a modified cabinet 102 such that front display opening 132 is wider and two of the three grow chambers 210 are displayed at a single time. Thus, first chamber 212 may be in the sealed position, while second chamber 214 and third chamber 216 may be in the display positions. As grow module 200 is rotated counterclockwise, first chamber 212 is moved into the display position and third chamber 216 is moved into the sealed position.

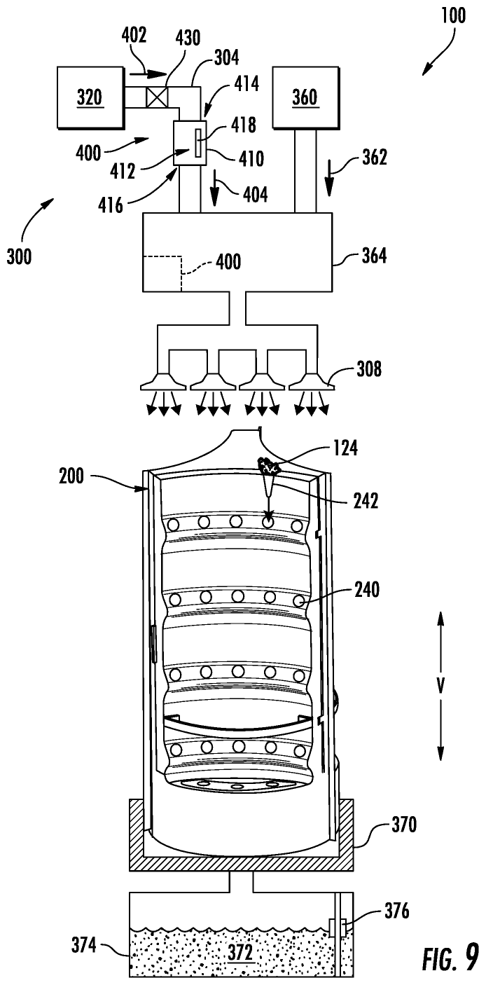

Referring now specifically to FIG. 9, gardening appliance 100 may further include a hydration system 300 that is generally configured for hydrating and/or sanitizing plants 124 within gardening appliance 100. In this regard, for example, hydration system 300 may be a part of or may entirely replace a hydration system 270 described above. Although an exemplary configuration and operation of hydration system 300 will be described below, it should be appreciated that variations and modifications may be made to such systems and methods while remaining within the scope of the present subject matter.

Although hydration system 300 is described herein as being used with gardening appliance 100, it should be appreciated that aspects of the present subject matter may be applied in any other suitable hydration system. For example, the hydration system 300 described herein may be used to provide hydrating water, nutrition, and/or sanitizing spray in any other suitable application, in any other suitable appliance, etc. In addition, variations and modifications may be made to the exemplary constructions described herein while remaining within the scope of the present subject matter.

According to the illustrated embodiment, hydration system 300 includes a supply conduit 304 and is generally configured for providing a flow of water and/or other nutrients into grow chamber 122 and/or root chamber 244. Specifically, hydration system 300 further includes one or more discharge nozzles 308 that are in fluid communication with supply conduit 304 to selectively provide the flow of liquid through discharge nozzles 308 to hydrate and/or sanitize plants 124. According to an exemplary embodiment, discharge nozzle 308 may be a part of or replace a hydration system 270 as illustrated in FIGS. 1 through 8. In this regard, discharge nozzle 308 may be equivalent to misting device 274 or may be used in addition to misting device 274. Although one exemplary configuration of discharge nozzle 308 is described herein, it should be appreciated that discharge nozzle 308 may include any other suitable number, type, configuration, and position of devices for supplying water, hydration, sanitizing spray, nutrients, etc. to plants 124.

According to exemplary embodiments, hydration system 300 may further include one or more valves positioned throughout hydration system 300 for regulating the flow of fluid therein. For example, as illustrated in FIG. 9, hydration system 300 includes a discharge valve 430 that is operably coupled to supply conduit 304 or directly to discharge nozzle 308 for selectively regulating the flow of liquid therethrough. Although a single discharge valve 430 is illustrated as regulating the flow of liquid to all discharge nozzles 308, it should be appreciated that hydration system 300 may include a plurality of independently adjustable discharge valves that can provide the flow of liquid to specific plants according to specific hydration schedules.

According to exemplary embodiments, supply conduit 304 may be fluidly coupled to any suitable number and type of fluid supplies to provide water through discharge nozzles 308. Specifically, according to an exemplary embodiment, hydration system 300 includes a water supply 320 for providing water such as pure tap water, distilled water, or water from any external fluid supply source. For example, water supply 320 may be a municipal water supply that provides a flow of pressurized water. According to still other embodiments, water supply 320 may include any other suitable sources of water, such as a water storage tank that may be filled by a user and that is contained within cabinet 102. It should be appreciated that water supply 320 may include any suitable pumps, flow regulating valves, or other flow regulating devices needed to regulate the flow of water.

According to exemplary embodiments, hydration system 300 may further include a nutrient dosing system 360 that is generally configured for facilitating the distribution of nutrient-rich liquid (identified herein generally by reference numeral 362) throughout gardening appliance 100 for improved plant growth. In this regard, for example, nutrient dosing system 360 may include a nutrient supply and a mixing system that provides a flow of nutrients 362 in the desired concentrations. Nutrient dosing system 360 may include replaceable nutrient cartridges that are filled with nutrients in concentrated form or may receive a nutrient supply from any other suitable location.

As used herein, the term “nutrients” and the like are intended generally to refer to any substances which facilitate improved growth of plants 124. For example, according to exemplary embodiments, nutrients may include calcium, magnesium, potassium, sulfur, copper, zinc, boron, molybdenum, iron, cobalt, manganese, phosphorous, and chlorine. Nutrients may also be used to refer to chemicals or substances that can be used to adjust a pH of the flow of liquid, a level of total dissolved solids (TDS), etc. According to alternative embodiments, any other suitable mixture or combination of compositions for encouraging root growth and plant growth may be used while remaining within the scope of the present subject matter.

Nutrient dosing system 360 may further include features for discharging selected flow rates or volumes of nutrients 362, such as pumps or discharge mechanisms. According to exemplary embodiments, nutrient dosing system 360 may include a plurality of solenoid-actuated plunger valves, a dedicated pump (e.g., such as a peristaltic pump), or a flow regulating valve that may selectively dispense any desired nutrients, at desired rates, and at desired times. Thus, nutrient dosing system 360 provides any suitable number, type, and combinations of nutrients 362 at any suitable flow rates and volumes for mixing within hydration system 300. For example, according to exemplary embodiments, nutrient dosing system 360 may include a plurality of flow regulating valves, discharge mechanisms, pumps, and supply nozzles that are all in operative communication with controller 174 of gardening appliance 100. As such, controller 174 may make informed decisions regarding the desired flow of diluted nutrient mixture based on the type, quality, and position of plants 124 within grow module 200. For example, controller 174 may regulate the type of nutrients supplied, the nutrient concentrations, which nozzles receive the flow of diluted nutrients, etc. In addition, nutrient dosing system 360 may make other adjustments that facilitate improved plant growth and ecosystem health within gardening appliance 100.

According to the illustrated embodiment, hydration system 300 may further include a mixing tank 364 that is generally configured for receiving water from water supply 320 along with nutrients 362 from nutrient dosing system 360. Mixing tank 364 may include any suitable agitators, stirrers, or other devices for creating a nutrient mixture out of nutrients 362 and water. Although nutrient dosing system 360 is illustrated as being fluidly coupled to hydration system 300 upstream mixing tank 364, it should be appreciated that nutrient dosing system 360 may be fluidly coupled to hydration system 300 in any other suitable location and in any other suitable manner.

Referring still to FIG. 9, in various embodiments, gardening appliance 100 may include a sanitization device or a sanitization assembly 400, such as a hypochlorous acid (HOCl) generator. Those of ordinary skill in the art will recognize that the HOCl generator 400 may be an electrolytic HOCl generator which catalyzes a reaction with chlorine in the water to produce the HOCl. As may be seen, e.g., in FIG. 9, the HOCl generator 400 may, in various embodiments, be positioned at any suitable location upstream of grow chamber 122. For example, according to the illustrated embodiment, HOCl generator may be integrated into hydration system 300 of gardening appliance 100. In this manner, HOCl generator 400 may be periodically activated to discharge a cleaning solution into grow chamber 122, as explained in more detail below. It should be appreciated that according to alternative embodiments, HOCl generator 400 may be an independent system from hydration system 300, e.g., including dedicated water supply conduits, control valves, discharge nozzles, etc.

Specifically, HOCl generator 400 may be fluidly coupled to a water supply (e.g., such as water supply 320) for receiving a flow of water (e.g., identified generally in FIG. 9 as flow of water 402). Specifically, according to the exemplary illustrated embodiment, HOCl generator 400 is integrated into hydration system 300, and is thus fluidly coupled to water supply 320 through water supply conduit 304. As will be explained in more detail below, when water 402 is provided to HOCl generator 400, a hypochlorous acid solution (e.g., identified generally in FIG. 9 by reference numeral 404) is generated that may be supplied onto plants 124 within grow chamber 122. As explained above, hypochlorous acid solution 404 may be a mild acidic solution that helps to kill bacteria, germs, fungi, and other potentially harmful or undesirable contaminants introduced onto the plants 124 during the growth process. In addition, the hypochlorous acid solution 404 is not harmful to humans and is safe for user interaction, contact, and consumption.

The HOCl generator 400 may be coupled to hydration system 300 in any suitable manner for receiving a flow of water (e.g., flow of water 402) and generating a flow of hypochlorous acid solution 404 upstream of grow chamber 122 for distribution onto plants 124. For example, as illustrated in solid lines in FIG. 9, the HOCl generator 400 is fluidly coupled to the water supply conduit 304 upstream of the mixing tank 364. In this manner, flow of water 402, whether supplied for hydration purposes, sanitization purposes, or both, passes through the HOCl generator 400 prior to entering mixing tank 364 where it may be mixed with nutrients and distributed into grow chamber 122. By activating HOCl generator 400 while the flow of water 402 is passing through the HOCl generator 400, hypochlorous acid may be generated in some concentration to generate hypochlorous acid solution 404.

According to an alternative embodiment, as shown in dotted lines in FIG. 9, the HOCl generator 400 may also be positioned within mixing tank 364. In this manner, by energizing HOCl generator 400 while water 402 is present within mixing tank 364, hypochlorous acid may be continually generated. Notably, this configuration may be used to obtain higher concentrations of hypochlorous acid within the hypochlorous acid solution 404 (e.g., as compared to the inline HOCl generator 400 described above), as the HOCl generator 400 has a longer interaction time with the water 402 that is stored at least temporarily within mixing tank 364.

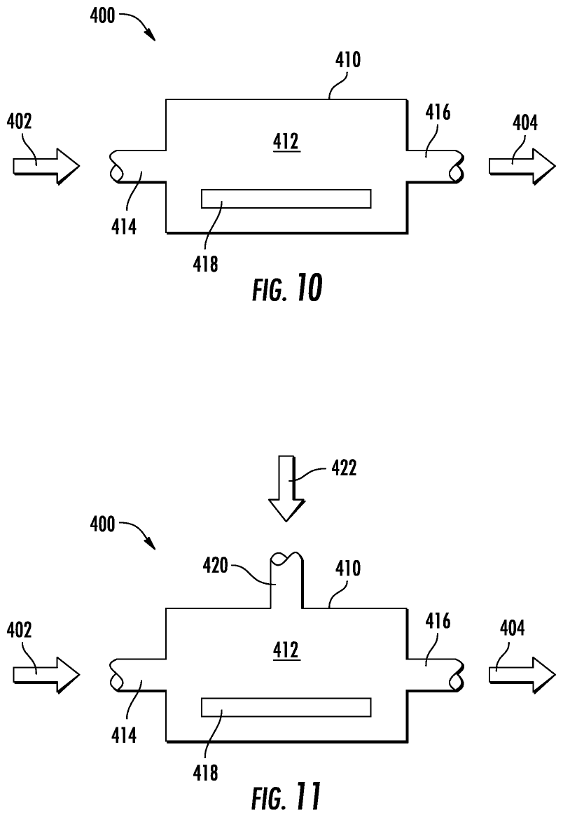

Referring now also to FIGS. 10 and 11, an exemplary HOCl generator 400 will be described according to exemplary embodiments of the present subject matter. As illustrated, the HOCl generator 400 may generally include a body 410 and a reaction chamber 412 defined in and by the body 410. As mentioned, an inlet 414 may be formed in or directly connected to the body 410 and the inlet 414 may be coupled to a water supply line whereby the flow of cold water 402 enters the electrolytic hypochlorous acid generator 400 from the water supply line at the inlet 414. In addition, an outlet 416 may be formed in or directly connected to the body 410 and the outlet 416 may be coupled to or positioned within mixing tank 364 for discharging the flow of hypochlorous acid solution 404 at the outlet 416.

An electrode 418 may be positioned in the reaction chamber 412 within the body 410. Thus, as those of ordinary skill in the art will understand, the electrode 418 may be activated, e.g., by providing a current thereto, and when so activated, the electrode 418 initiates or catalyzes a reaction among constituent elements in the flow of cold water 402, including solutes and other substances therein, such as chlorine, to form or generate HOCl solution 404 within the reaction chamber 412. The HOCl generator 400 may discharge the HOCl solution 404, e.g., including water, HOCl, and/or other substances, through outlet 416 downstream of the reaction chamber 412.

In particular, some embodiments, e.g., the exemplary embodiment illustrated in FIG. 10, of the electrolytic HOCl generator 400 include only a single inlet 414 and no other points of ingress or inflow into the reaction chamber 412, whereas alternative embodiments, e.g., as illustrated in FIG. 11, include a second inlet 420. The inlet 414 may be coupled to and/or in fluid communication with a water supply line, whereby the inlet 414 receives a flow of cold water from the water supply line, e.g., directly from the water supply line (FIG. 9). Thus, in embodiments where the inlet 414 is the only inlet into the reaction chamber 412, the electrolytic HOCl generator 400 uses chlorine which is already present in the cold water, e.g., a background or baseline chlorine concentration such as residual chlorine from a water treatment system upstream of gardening appliance 100, to generate the HOCl. In embodiments which include an additional inlet, e.g., inlet 420, the electrolytic HOCl generator 400 may receive an additive including a reactant which may then used to create additional HOCl.

As discussed above, in some embodiments, the inlet 414 of the electrolytic hypochlorous acid generator 400 that is coupled to the water supply line is the only inlet into the reaction chamber 412 of the electrolytic hypochlorous acid generator 400, whereby the reaction which produces the HOCl in the reaction chamber 412 consumes only background chlorine already present in the cold water 402. In alternative embodiments, e.g., as illustrated in FIG. 11, the electrolytic HOCl generator 400 may receive an additive, e.g., a reactant, 422 from a reactant supply (e.g., a reservoir, which is not specifically illustrated or described in further detail because the structure and function of reservoirs would be understood by those of ordinary skill in the art) into the reaction chamber 412 which reacts with the cold water and/or adjusts the conditions, such as pH, within the reaction chamber 412 when the electrode 418 is activated, thereby resulting in a higher concentration of HOCl, e.g., a higher parts per million (ppm) of HOCl, in the flow of HOCl solution 404 in embodiments with multiple inlets into the reaction chamber 412 within the body 410 of the electrolytic HOCl generator 400.

For example, the additive 422 may be a compound including table salt, e.g., sodium chloride or NaCl, water (H2O), and a mild acid such as vinegar, e.g., acetic acid or CH3COOH. The pH of the solution (e.g., tap water and additive mixed together) within the reaction chamber 412 correlates to the product of the reaction when the electrode 418 is activated, and the additive may help provide a pH which is high enough to avoid generating chlorine gas (Cl2) and low enough to avoid producing bleach, e.g., sodium hypochlorite (NaOCl or NaClO) and/or hypochlorite ions (OCl— or ClO—), whereby HOCl is generated preferentially to chlorine gas or bleach when the reaction occurs at the desired pH, such as generating at least about 90% HOCl.

For example, the desired pH may be generally in a range that those of ordinary skill in the art will recognize as being weakly acidic, such as between about 3.0 and about 6.5, such as between about 4.0 and about 5.5. For example, a pH of about 3.0 may result in a production of about 10% Cl2 and about 90% HOCl, whereas a pH of about 6.5 may result in a production of about 10% bleach (hypochlorite ions and/or hypochlorite salt such as sodium hypochlorite) and about 90% HOCl, and pH values between 3.0 and 6.5 may generate at least about 90% HOCl. In particular, a pH of the solution between about 4.0 and about 5.5 may generate about 97% HOCl or higher, such as about 99% HOCl, such as about 100% HOCl (where the stated percentages are relative to other chlorine species, e.g., chlorine gas or bleach, as discussed herein).

Embodiments which include an additional inlet 420 for receiving additive may advantageously provide stronger disinfection due to the higher ppm of HOCl in the flow of HOCl solution 404. However, such embodiments may also be more sensitive to the pH of the solution. For example, the higher levels of chlorine provided by the additive may result in generation of undesirable quantities of chlorine gas if the solution is too acidic (pH is too low) or hypochlorite ions if the solution is too basic (pH too high) instead of the desired HOCl. Embodiments which include only the single inlet coupled to the water supply line advantageously provide a simpler structure, not only of the HOCl generator 400 itself, but of the sanitization system overall, e.g., in that the additive reservoir and associated pump or injection system is not required or included in such embodiments. Further, in the single-inlet embodiments although the proportion of chlorine present in the tap water (the flow of cold water 402) is relatively low, e.g., as compared to the level of chlorine in the additive 422 in other embodiments which include the second inlet 420, the relatively large volume of the water 402 (again, as compared to the volume of additive 422) generally provides sufficient total chlorine for sanitizing the laundry appliance.

As explained above, according to the embodiment illustrated in FIG. 9, inlet 414 may be fluidly coupled to a source of water, e.g., water supply 320. For example, as illustrated in FIG. 9, inlet 414 may be fluidly coupled to water supply conduit 304 (e.g., as shown in solid lines) or may be positioned within mixing tank 364 (e.g., as shown in dotted lines) to circulate water 402 from within mixing tank 364 through HOCl generator 400 to increase a concentration of hypochlorous acid within the HOCl solution 404.

According to exemplary embodiments, the flow of water that passes into mixing tank 364 and/or through HOCl generator 400 may be regulated by a valve 430, as shown schematically in FIG. 9. For example, valve 430 may be a supply valve and, in at least some embodiments a cold water supply valve. Controller 174 of gardening appliance 100 (or another suitable dedicated controller) may be configured for selectively opening valve 430 to provide the flow of water 402, e.g., cold water, into gardening appliance 100. Thus, the valve 430 may be upstream of other components of the gardening appliance 100, in particular the HOCl generator 400, such that selective operation of valve 430 and/or HOCl generator 400 may regulate the generation of hypochlorous acid. The structure and function of such valves are understood by those of ordinary skill in the art and, as such, are not shown or described in further detail herein for the sake of brevity and clarity.

As mentioned, the water which flows through gardening appliance 100 may be or include cold water. Cold water may include water having a temperature based on the water source(s) from which the water is obtained. Further, “cold” water as used herein to include a wide range of temperatures that are not hot. For example, cold water, as used herein, may be between approximately 35 degrees Fahrenheit and approximately 120 degrees Fahrenheit, such as between approximately 40 degrees Fahrenheit and approximately 110 degrees Fahrenheit, such as between approximately 45 degrees Fahrenheit and approximately 100 degrees Fahrenheit, such as between approximately 55 degrees Fahrenheit and approximately 90 degrees Fahrenheit, such as between approximately 65 degrees Fahrenheit and approximately 80 degrees Fahrenheit, such as between approximately 40 degrees Fahrenheit and approximately 60 degrees Fahrenheit, such as approximately 50 degrees Fahrenheit, or approximately 80 degrees Fahrenheit, or approximately 110 degrees Fahrenheit.

Thus, when the HOCl generator 400 is activated, e.g., by supplying electric power thereto such as in embodiments where the HOCl generator 400 is an electrolytic HOCl generator, the liquid flowing therethrough will include hypochlorous acid (HOCl), such as a solution of water and HOCl 404. Further, it should be understood that the liquid, e.g., “water,” may also include additional constituents, e.g., nutrients, minerals, chemicals, and other substances.

Referring still to FIG. 9, hydration system 300 may further include features for collecting, discharging, and/or recirculating the flow of HOCl solution 404 or other liquid within grow chamber 122. In this regard, plants 124 may not absorb all of the flow of liquid provided from hydration system 300 dispensed from discharge nozzles 308. Therefore, the excess liquid may drip off of plants 124 and collect at the bottom of gardening appliance 100. Thus, according to the illustrated embodiment, hydration system 300 includes a sump 370 that is generally configured for collecting liquid (e.g., referred to herein as drainage water 372) from within grow chamber 122.

Hydration system 300 may further include a wastewater reservoir or tank 374 that is fluidly coupled to sump 370 for storing drainage water 372. According to exemplary embodiments, wastewater reservoir 374 may be removable from gardening appliance 100, such that a user may periodically empty or drain wastewater reservoir 374 through an external drain, such as a kitchen sink. According to still other embodiments, wastewater reservoir 374 may be connected to an external drain via a flow regulating valve and/or a drainage pump for periodically or selectively discharging drainage water 372 from wastewater reservoir 374.

Notably, in order to prevent overfilling of wastewater reservoir 374, gardening appliance 100 may include features for ensuring that the available storage capacity of wastewater reservoir 374 is sufficient for storing liquid generated during a sanitization cycle prior to initiation of the sanitization cycle. As such, as best shown in FIG. 9, wastewater reservoir 374 may further include a water level sensor 376 that is positioned within wastewater reservoir 374 for measuring a level of drainage water 372 therein. In this manner, feedback from water level sensor 376 may be used to determine when a sanitization cycle may be performed without risking the overflow of wastewater reservoir 374. Level sensor 376 can be any suitable type of sensor, such as a float switch, an optical switch, a capacitance-based level sensor, etc.

In general, controller 174 of gardening appliance 100 may be configured for regulating operation of HOCl generator 400 and a hydration system 300. In this regard, controller 174 may be configured to receive a command to commence a sanitation cycle. As used herein, the terms “sanitization cycle” and the like are generally intended to refer to any period of operation when HOCl generator 400 is generating a flow of HOCl solution 404 to facilitate a cleaning or sanitization cycle of plants 124 or other surfaces within gardening appliance 100.

According to exemplary embodiments, the command to commence a sanitization cycle may be received from a user of gardening appliance 100. In this regard, for example, control panel 170 may include a user input 172 that a user may select to initiate a sanitization cycle. According to exemplary embodiments, controller 174 may be in operative communication with a remote device, such as a mobile phone running a software application that may be used to input the command to commence a sanitization cycle. According to alternative embodiments, the command to commence a sanitization cycle may be generated periodically by the controller 174 or may otherwise be operated according to a preprogrammed schedule that may be manipulated by a user, set by a manufacturer, etc. This sanitization cycle is generally intended for maintaining the plants in a clean growing environment within gardening appliance 100.

Notably, as displayed above, controller 174 may perform a detection cycle to ensure that the storage capacity within wastewater reservoir 374 is sufficient to store the additional liquid that is generated during a sanitization cycle. As such, when controller receives the command to commence a sanitization cycle, controller 174 may first determine that the storage capacity is sufficient to store the hypochlorous acid that will be generated during the sanitization cycle, e.g., by using the level sensor 376 and the known capacity of wastewater reservoir 374.

Once it is determined that there is sufficient capacity to store the excess liquid generated during a sanitization cycle, controller 174 may be programmed to initiate the sanitization cycle, e.g., by opening valve 430 to provide the flow of water 402 into HOCl generator 400. In addition, controller 174 may energize the electrode 418 or otherwise activate HOCl generator 400 such that hypochlorous acid is generated from the flow of water 402. This hypochlorous acid and the excess water from flow of water 402 generally form an HOCl solution 404 which is then discharged onto plants 124 in a manner similar to hydration cycles, e.g., via discharge nozzles 308.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. An indoor gardening appliance, comprising:
a liner positioned within a cabinet and defining a grow chamber;
a grow module mounted within the liner and defining a plurality of apertures for receiving one or more plant pods; and
a sanitization assembly comprising:
a water supply for providing a flow of water;
an electrolytic hypochlorous acid generator for receiving the flow of water and generating a hypochlorous acid solution; and
a discharge nozzle for selectively discharging the hypochlorous acid solution into the grow chamber.

2. The indoor gardening appliance of claim 1, wherein the electrolytic hypochlorous acid generator comprises:
a body defining a reaction chamber, an inlet fluidly coupled to the water supply, and an outlet fluidly coupled to the discharge nozzle; and
an electrode positioned in the reaction chamber.

3. The indoor gardening appliance of claim 2, wherein the sanitization assembly further comprises:
a valve for regulating the flow of water through the reaction chamber.

4. The indoor gardening appliance of claim 3, wherein the valve is positioned on a water supply conduit upstream of the electrolytic hypochlorous acid generator.

5. The indoor gardening appliance of claim 3, further comprising a controller operably coupled to the valve and the electrolytic hypochlorous acid generator, the controller being configured to:
receive a command to commence a sanitization cycle;
open the valve to provide the flow of water into the reaction chamber; and
energize the electrode to generate the hypochlorous acid solution.

6. The indoor gardening appliance of claim 5, wherein the command to commence the sanitization cycle is generated periodically by the controller.

7. The indoor gardening appliance of claim 5, further comprising a user interface panel, wherein the command to commence the sanitization cycle is selected by a user through the user interface panel or a software application on a remote device.

8. The indoor gardening appliance of claim 5, wherein the controller is further configured to:
determine that a wastewater reservoir has storage capacity to store the hypochlorous acid solution generated during the sanitization cycle before commencing the sanitization cycle.

9. The indoor gardening appliance of claim 1, further comprising a hydration system that comprises the sanitization assembly, the hydration system further comprising:
a mixing tank fluidly coupled to the water supply through a water supply conduit; and
a nutrient dosing system for selectively adding nutrients to the mixing tank for creating a nutrient mixture within the mixing tank.

10. The indoor gardening appliance of claim 9, wherein the electrolytic hypochlorous acid generator is fluidly coupled to the water supply conduit upstream of the mixing tank.

11. The indoor gardening appliance of claim 9, wherein the electrolytic hypochlorous acid generator is positioned within the mixing tank.

12. The indoor gardening appliance of claim 9, wherein the hydration system further comprises:
a sump positioned at a bottom of the grow chamber for collecting drainage water; and
a wastewater reservoir fluidly coupled to the sump for storing the drainage water.

13. The indoor gardening appliance of claim 1, wherein the water supply comprises a municipal water supply or a water reservoir.

14. A hydration system for a gardening appliance, the gardening appliance comprising a liner positioned within a cabinet and defining a grow chamber and a grow module mounted within the liner and defining a plurality of apertures for receiving one or more plant pods, the hydration system comprising:
a water supply for providing a flow of water;
a mixing tank fluidly coupled to the water supply through a water supply conduit; and
a sanitization assembly comprising:
an electrolytic hypochlorous acid generator fluidly coupled to the mixing tank for receiving the flow of water and generating a hypochlorous acid solution; and
a discharge nozzle for selectively discharging the hypochlorous acid solution into the grow chamber.

15. The hydration system of claim 14, further comprising:
a nutrient dosing system for selectively adding nutrients to the mixing tank for creating a nutrient mixture within the mixing tank.

16. The hydration system of claim 14, wherein the electrolytic hypochlorous acid generator comprises:
a body defining a reaction chamber, an inlet fluidly coupled to the water supply, and an outlet fluidly coupled to the discharge nozzle; and
an electrode positioned in the reaction chamber.

17. The hydration system of claim 14, wherein the electrolytic hypochlorous acid generator is fluidly coupled to the water supply conduit upstream of the mixing tank.

18. The hydration system of claim 14, wherein the electrolytic hypochlorous acid generator is positioned within the mixing tank.

19. The hydration system of claim 14, further comprising:
a valve for regulating the flow of water through the electrolytic hypochlorous acid generator.

20. The hydration system of claim 19, further comprising a controller operably coupled to the valve and the electrolytic hypochlorous acid generator, the controller being configured to:
receive a command to commence a sanitization cycle;
open the valve to provide the flow of water into the electrolytic hypochlorous acid generator; and
operate the electrolytic hypochlorous acid generator to generate the hypochlorous acid solution.
